(19) United States
(12) Patent Application Publication
(10) Pub. No.: US 2017/0128709 A1
(43) Pub. Date: May 11, 2017

(54) Venous Access Implantable Port

(71) Applicant: Yifang Chen, Ann Arbor, MI (US)

(72) Inventor: Yifang Chen, Ann Arbor, MI (US)

(21) Appl. No.: 15/345,504

(22) Filed: Nov. 7, 2016

Related U.S. Application Data
(60) Provisional application No. 62/251,910, filed on Nov. 6, 2015.

(51) Int. Cl. A61M 39/02

(52) U.S. Cl. CPC A61M 39/0247; A61M 2039/0202; A61M 2039/0258; A61M 2039/0261; A61M 2039/0264; A61M 2039/0273; A61M 2039/0276

(57) ABSTRACT

A venous access implantable port allows a doctor or medical practitioner to use the same injection or extraction site for multiple procedures. The venous access implantable port includes a port body, a venous access orifice, a fluid reservoir, at least one fluid transport tube, and a catheter. The port body is transdermally positioned at the procedural location. The venous access orifice traverses into the port body in order to allow a needle to be inserted through the venous access orifice to extract or inject fluid into the fluid reservoir integrated into the port body. The fluid reservoir is in fluid communication with a patient's circulatory system through the catheter and the at least one fluid transport tube traversing into the port body, in order to allow the extraction or injection of fluids into the circulatory system.

The current application claims a priority to the U.S. Provisional Patent application Ser. No. 62/251,910 filed on Nov. 6, 2015. The current application is filed on Nov. 7, 2016 while Nov. 6, 2016 was on a weekend.

### FIELD OF THE INVENTION

The present invention relates generally to a venous medical device. More specifically, the present invention relates to a venous access port to provide access to a catheter. The present invention has a cuff integrated around the port. A new way and concept of placement of the port: the port septum outside of skin and the port with a polyethylene terephthalate cuff.

### BACKGROUND OF THE INVENTION

The standard venous access implantable ports secured to an implanted catheter for the infusion and/or withdrawal of fluids from a patient are well-known and for a long term use. These ports are typically used for drug infusion or for withdrawal of small amounts of blood, where large flows of fluid are not required. The ports are assemblies of a needle-impenetrable housing with a discharge port in fluid communication with the catheter and the reservoir within the port housing, and the ports provide a subcutaneous self-sealing septum that defines an access site for multiple needle sticks through the covering skin tissue of the patient, through the septum and into the reservoir, without the need to continuously search for new venous access sites. A venous access port assembly is desired to provide a medical practitioner with capability to discern an important property of the port assembly after the port assembly has been implanted into a patient.

The present invention is a venous access implantable port to be inserted transdermally. The present invention provides access to treatment or surgical sites through a catheter. The catheter is integrated into the circulatory system in order to draw blood from or infuse drugs into the circulatory system. The present invention allows for multiple insertions for needles at the same site, and simultaneously allows a doctor or medical practitioner to easily find the present invention.

### BRIEF DESCRIPTION OF THE DRAWINGS

FIG. 1 is a perspective view of the present invention.

FIG. 2 is a side view of the present invention, wherein the subcutaneous mounting overlay is removed.

FIG. 3 is a top view of the present invention, wherein the subcutaneous mounting overlay is removed.

FIG. 4 is a cross-sectional view of the present invention along the line 4-4 from FIG. 3, wherein the subcutaneous mounting overlay is included.

FIG. 5 is a perspective view of an alternate embodiment of the present invention, wherein the subcutaneous mounting overlay is removed.

FIG. 6 is a side view of the alternate embodiment of the present invention.

FIG. 7 is a cross-sectional view of the alternate embodiment present invention along the line 7-7 from FIG. 6.

FIG. 8 is a perspective view of another alternate embodiment of the present invention, wherein the subcutaneous mounting overlay is removed.

FIG. 9 is a side view of the other alternate embodiment of the present invention.

FIG. 10 is a cross-sectional view of the other alternate embodiment present invention along the line 10-10 from FIG. 9.

### DETAIL DESCRIPTIONS OF THE INVENTION

All illustrations of the drawings are for the purpose of describing selected versions of the present invention and are not intended to limit the scope of the present invention.

The present invention is a venous access implantable port. The present invention allows for a reusable site for extracting blood samples or infusing drugs for the treatment of a patient. The present invention is transdermally positioned on a patient in order to allow a doctor or another medical professional to access the present invention efficiently, while allowing the present invention to interface with a vein of a patient's circulatory system.

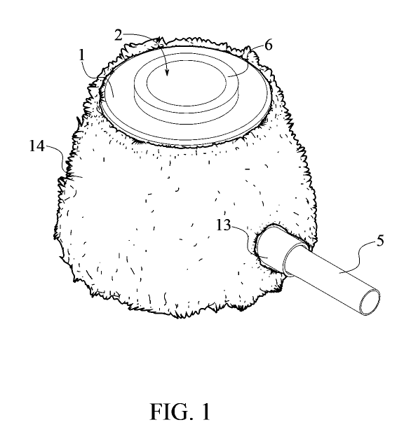

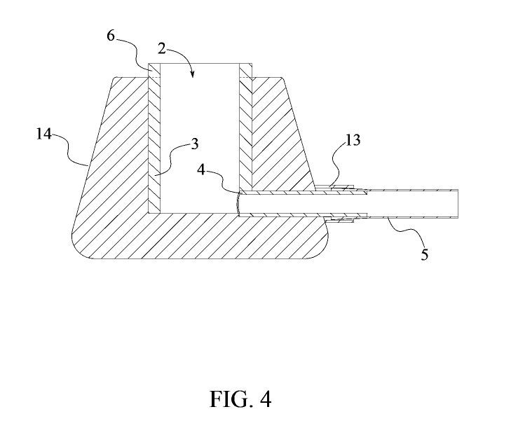

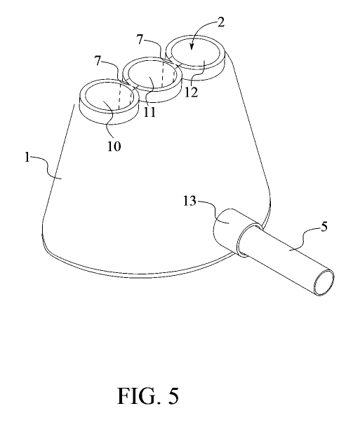

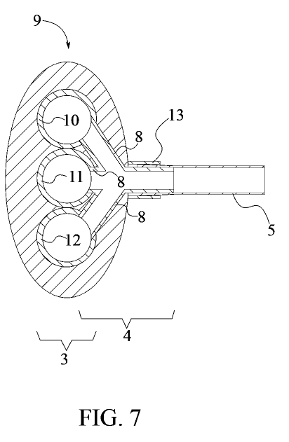

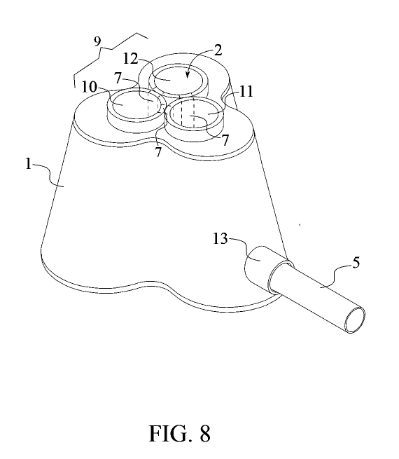

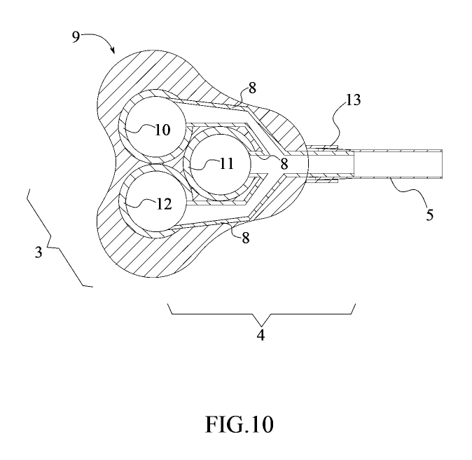

In accordance to FIG. 4, the present invention comprises a port body 1, a venous access orifice 2, a fluid reservoir 3, at least one fluid transport tube 4, and a catheter 5. The port body 1 provides structure to the present invention in order to prevent the present invention from being crushed under the pressure from the surrounding skin, as the present invention is positioned transdermally during implementation. The venous access orifice 2 traverses into the port body 1, as shown in FIG. 1, FIG. 4, FIG. 5 and FIG. 8. The venous access orifice 2 allows a doctor or medical professional to insert a needle into the present invention in order to inject or extract fluids into or from the patient's circulatory system. The fluid reservoir 3 retains a quantity of fluid in order to prevent air from being introduced into the blood stream as fluid is being injected or extracted through the present invention. The fluid reservoir 3 is integrated into the port body 1, as shown in FIG. 4, FIG. 7, and FIG. 10. The venous access orifice 2 is in fluid communication with the fluid reservoir 3 in order to allow fluid to be transferred between a needle inserted through the venous access orifice 2 and the fluid reservoir 3. The at least one fluid transport tube 4 allows fluids to be transferred between the fluid reservoir 3 and the catheter 5. The at least one fluid transport tube 4 laterally traverses into the port body 1, and the at least one fluid transport tube 4 traverses into the fluid reservoir 3 from the port body 1. The catheter 5 directly interfaces with the patient's circulatory system in order to deliver or extract fluid from the patient. The catheter 5 is laterally positioned with the port body 1. The fluid reservoir 3 is in fluid communication with the catheter 5 through the at least one fluid transport tube 4 in order to allow fluid to be transferred between the patient the fluid reservoir 3.

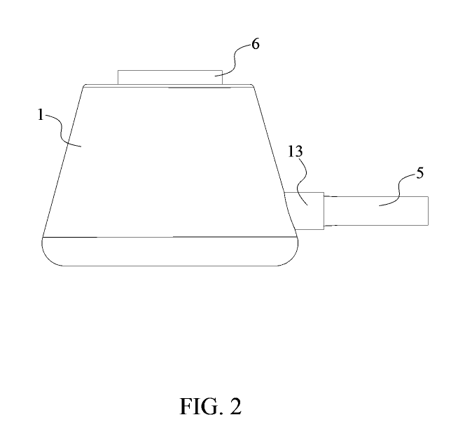

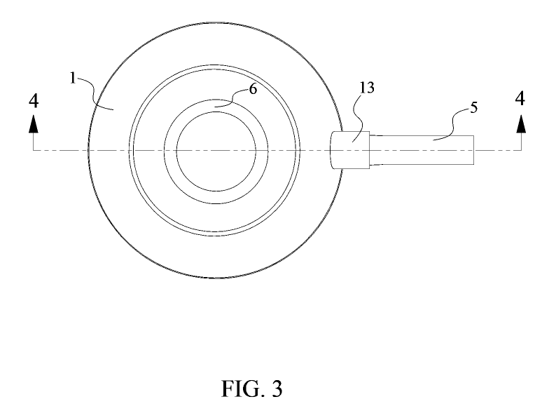

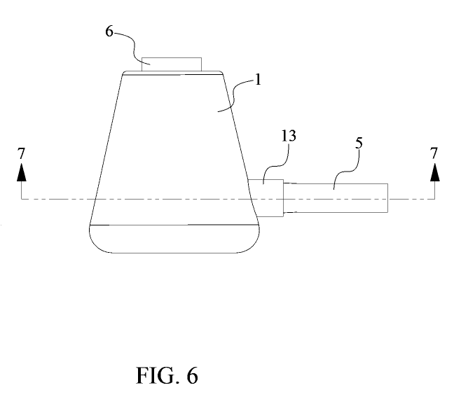

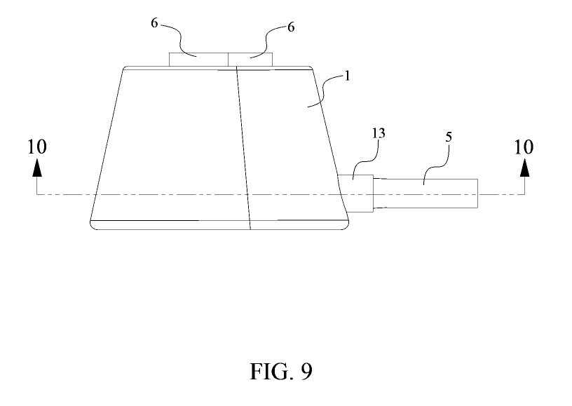

In accordance to the preferred embodiment of the present invention, the present invention comprises an extruded cuff 6, as detailed in FIG. 1 to FIG. 4, FIG. 6, and FIG. 9. The extruded cuff 6 extends out from the patient's skin. The extruded cuff 6 allows needles to be more easily inserted to the present invention. The extruded cuff 6 is externally connected to the port body 1. The extruded cuff 6 is perimetrically positioned about the venous access orifice 2 in order to help a doctor or medical practitioner to insert a needle into the present invention.

In some embodiments of the present invention, the present invention comprises at least one septum 7, and the at least one fluid transport tube 4 is a plurality of fluid transport tubes 8 in accordance to FIG. 7 and FIG. 10. The at least one septum 7 partitions the fluid reservoir 3 into a plurality of lumina 9. The plurality of lumina 9 is a series of hollow structures for fluid delivery or extraction between a needle inserted into a lumen of the plurality of lumina 9 and the at least one fluid transport tube 4. Each of the fluid transport tubes of the plurality of transport tubes is in fluid communication with a corresponding lumen of the plurality of lumina 9 to provide multiple fluid channels to and from the catheter 5.

In a more specific embodiment of the present invention, the first lumen 10 is adjacently positioned with the second lumen 11, and the third lumen 12 is adjacently positioned with the second lumen 11, as shown in FIG. 5 and FIG. 7. The first lumen 10 is oppositely positioned with the third lumen 12 about the second lumen 11 such that the first lumen 10, the second lumen 11 and the third lumen 12 are linearly aligned to allow the doctor or medical practitioner to designate each of the first lumen 10, the second lumen 11, and the third lumen 12 for a specific purpose. In an alternate specific embodiment, the plurality of lumina 9 comprises a first lumen 10, a second lumen 11, and a third lumen 12, as shown in FIG. 8 and FIG. 10. The first lumen 10 is adjacently positioned with the second lumen 11 and the third lumen 12, and the second lumen 11 is adjacently positioned with the first lumen 10 and the third lumen 12. This configuration provides the doctor or medical practitioner with equal accessibility to the first lumen 10, the second lumen 11, and the third lumen 12.

Further in accordance to the preferred embodiment, the present invention comprises a catheter lock 13, as shown in FIG. 1 to FIG. 10. The catheter lock 13 secures the catheter 5 to the port body 1. The catheter lock 13 is externally and laterally connected to the port body 1. The catheter lock 13 is positioned about the at least one fluid transport tube 4. The catheter lock 13 is selectively engaged with the catheter 5 in order to allow the positioning procedure for the catheter 5 to be performed independent of the insertion or removal of the port body 1 into the patient's skin.

The present invention further comprises a subcutaneous tissue mounting overlay 14, in accordance to FIG. 1 and FIG. 4. The subcutaneous tissue mounting overlay 14 is a coating or covering to adhere the present invention to the subcutaneous tissue in order to retain the transdermal position of the port body 1. The subcutaneous tissue mounting overlay 14 is laterally superimposed onto the port body 1 such that the subcutaneous tissue mounting overlay 14 is able to interface with the patient's subcutaneous tissue to secure the positioning of the port body 1. The subcutaneous tissue mounting overlay 14 is preferred to be polyethylene terephthalate, also known as Darcon, to graft the present invention to the subcutaneous tissue.

Although the invention has been explained in relation to its preferred embodiment, it is to be understood that many other possible modifications and variations can be made without departing from the spirit and scope of the invention as hereinafter claimed.

What is claimed is:

1. A venous access implantable port comprises:
a port body;
a venous access orifice;
a fluid reservoir;
at least one fluid transport tube;
a catheter;
the venous access orifice traversing into the port body;
the fluid reservoir being integrated into the port body;
the at least one fluid transport tube laterally traversing into the port body;
the at least one fluid transport tube traversing into the fluid reservoir from the port body;
the catheter being laterally positioned with the port body;
the venous access orifice being in fluid communication with the fluid reservoir; and
the fluid reservoir being in fluid communication with the catheter through the at least one fluid transport tube.

2. The venous access implantable port, as claimed in claim 1, comprises:
an extruded cuff;
the extruded cuff being externally connected to the port body; and
the extruded cuff being perimetrically positioned about the venous access orifice.

3. The venous access implantable port, as claimed in claim 1, comprises:
at least one septum;
the at least one fluid transport tube being a plurality of fluid transport tubes;
the fluid reservoir being partitioned by the at least one septum into a plurality of lumina; and
each of the fluid transport tubes of the plurality of fluid transport tubes being in fluid communication with a corresponding lumen of the plurality of lumina.

4. The venous access implantable port, as claimed in claim 3, comprises:
the plurality of lumina being a first lumen, a second lumen, and a third lumen;
the first lumen being adjacently positioned with the second lumen;
the third lumen being adjacently positioned with the second lumen; and
the first lumen being oppositely positioned to the third lumen about the second lumen.

5. The venous access implantable port, as claimed in claim 3, comprises:
the plurality of lumina comprises a first lumen, a second lumen, and a third lumen;
the first lumen being adjacently positioned with the second lumen and the third lumen; and
the second lumen being adjacently positioned with the first lumen and the third lumen.

6. The venous access implantable port, as claimed in claim 1, comprises:
a catheter lock;
the catheter lock being externally and laterally connected to the port body;
the catheter lock being positioned about the at least one fluid transport tube; and
the catheter lock being selectively engaged with the catheter.

7. The venous access implantable port, as claimed in claim 1, comprises:
a subcutaneous tissue mounting overlay; and
the subcutaneous tissue mounting overlay being laterally superimposed onto the port body.

8. A venous access implantable port comprises:
a port body;
a venous access orifice;
a fluid reservoir;
at least one fluid transport tube;
a catheter;
an extruded cuff;
the venous access orifice traversing into the port body;
the fluid reservoir being integrated into the port body;
the at least one fluid transport tube laterally traversing into the port body;
the at least one fluid transport tube traversing into the fluid reservoir from the port body;
the catheter being laterally positioned with the port body;
the venous access orifice being in fluid communication with the fluid reservoir;
the fluid reservoir being in fluid communication with the catheter through the at least one fluid transport tube;
the extruded cuff being externally connected to the port body; and
the extruded cuff being perimetrically positioned about the venous access orifice.

9. The venous access implantable port, as claimed in claim 8, comprises:
at least one septum;
the at least one fluid transport tube being a plurality of fluid transport tubes;
the fluid reservoir being partitioned by the at least one septum into a plurality of lumina; and
each of the fluid transport tubes of the plurality of fluid transport tubes being in fluid communication with a corresponding lumen of the plurality of lumina.

10. The venous access implantable port, as claimed in claim 9, comprises:
the plurality of lumina being a first lumen, a second lumen, and a third lumen;
the first lumen being adjacently positioned with the second lumen;
the third lumen being adjacently positioned with the second lumen; and
the first lumen being oppositely positioned to the third lumen about the second lumen.

11. The venous access implantable port, as claimed in claim 9, comprises:
the plurality of lumina comprises a first lumen, a second lumen, and a third lumen;
the first lumen being adjacently positioned with the second lumen and the third lumen; and
the second lumen being adjacently positioned with the first lumen and the third lumen.

12. The venous access implantable port, as claimed in claim 8, comprises:
a catheter lock;
the catheter lock being externally and laterally connected to the port body;
the catheter lock being positioned about the at least one fluid transport tube; and
the catheter lock being selectively engaged with the catheter.

13. The venous access implantable port, as claimed in claim 8, comprises:
a subcutaneous tissue mounting overlay; and
the subcutaneous tissue mounting overlay being laterally superimposed onto the port body.
